(19) United States
(12) Patent Application Publication
(10) Pub. No.: US 2021/0304895 A1
(43) Pub. Date: Sep. 30, 2021

(54) SYSTEM AND METHOD FOR GENERATING CURATED INTERVENTIONS IN RESPONSE TO PATIENT BEHAVIOR

(71) Applicant: TapRoot Interventions & Solutions, Inc., Phoenix, AZ (US)

(72) Inventors: Linda S. Buscemi, Phoenix, AZ (US); David Schneider, Phoenix, AZ (US); Scarlett Spring, Phoenix, AZ (US)

(21) Appl. No.: 17/213,164

(22) Filed: Mar. 25, 2021

Related U.S. Application Data
(60) Provisional application No. 63/000,625, filed on Mar. 27, 2020.

(51) Int. Cl. G16H 50/20; G16H 40/67; G16H 10/20; G16H 70/20; G16H 50/70; G16H 15/00; G16H 10/60; G06N 20/20

(52) U.S. Cl. CPC G16H 50/20; G16H 40/67; G16H 10/20; G06N 20/20; G16H 50/70; G16H 15/00; G16H 10/60; G16H 70/20

(57) ABSTRACT

Embodiments of the present disclosure provide for methods, system, and architecture for automatically generating curated interventions for modifying patient behaviors within an end user application is disclosed herein. A patient behavior is observed including a set of features. A computer program stored on a non-transitory computer-readable medium is used to select the patient and the observed patient behavior. The selected patient and the selected patient behavior are loaded into a trained intervention generating system. The trained intervention generating system generates an intervention responsive to the selected patient and the selected patient behavior inputs using artificial intelligent neural networks. The intervention recommender system is accessible to caregiving users via an interactive cloud computing architecture and service incorporating sensors, camera, Internet of Things technology for monitoring patient behaviors and daily activities.

### CROSS-REFERENCE TO RELATED APPLICATIONS

This application claims priority benefit of U.S. Provisional Application Ser. No. 63/000,625 filed Mar. 27, 2020, the entirety of which is incorporated herein at least by virtue of this reference.

### FIELD

The present invention relates to a system and method for automatically generating curated interventions in response to patient behavior. More particularly, the invention contemplates implementation for those aiding and caring for individuals with behaviors related to diseases such as dementia and/or Alzheimer's disease, mental health behaviors such as personality disorders, autism, Asperger's and/or other cognitive impairments.

### BACKGROUND

Behavioral healthcare is reliant on a systematic approach to identify, diagnose, and treat issues in a reliable, predictable, and consistent manner. Using a structured systematic approach accommodates numerous benefits ranging from analysis of post-treatment care to phenotypic subgroup treatment plans and so on. Structured approaches require an ontology upon which to work.

Ontological modeling is the process to explicitly specify key concepts and their properties for a problem domain. These concepts are organized in a hierarchical structure through their shared properties to form superclass or categories and subclass relations. Computational behavorial models are required in order to perform behavior activity recognition. Currently, there is a lack of technological framework (e.g., intelligence assistance) in the behavior and mental health space describing the relationship and interconnectedness between encounter data, patient behaviors, suggestion interventions, and outcome. The current state of the art has been deficient in developing a formal ontology in this field defined by categories and subcategories (i.e., taxonomies) as well as folksonomies (i.e., a way of organizing data and digital content). In addition, a model which merely predicts an intervention from behavior is insufficient since it does not consider the required time, resources, effort, and effectiveness of an intervention. Therefore, conventional behavorial intervention predictive models lack caregiver action efficiency as a key element of an optimal intelligent assistance system for providing care to persons living with mental and behavior disorders or impairments.

Through applied effort, ingenuity, and innovation, Applicant has identified a number of deficiencies and problems with technology-based solutions to assist caregivers in managing and caring for persons living with mental or behavior health disorders or impairments. Applicant has developed a solution that is embodied by the present invention, which is described in detail below.

### SUMMARY

The following presents a simplified summary of some embodiments of the invention in order to provide a basic understanding of the invention. This summary is not an extensive overview of the invention. It is not intended to identify key/critical elements of the invention or to delineate the scope of the invention. Its sole purpose is to present some embodiments of the invention in a simplified form as a prelude to the more detailed description that is presented later.

A computer implemented method and system for automatically generating curated interventions for modifying patient behaviors in a patient is disclosed herein. A patient behavior is observed including a set of features. A computer program is used to select the patient and the observed patient behavior from a table. The selected patient and the selected patient behavior are loaded into a trained intervention generating system. The trained intervention generating system generates an intervention responsive to the selected patient and the selected patient behavior inputs. In one embodiment, a success level is recorded for the intervention. In an alternative embodiment the amount of time, required resources, frequency, intensity and effectiveness of the intervention are recorded for the intervention. A training set including the selected patient, the selected patient behavior inputs, and the success level, amount of time, required resources, frequency, intensity, and intervention effectiveness is built and used to update training of the intervention generating system. Outcome data are provided based on a plurality of factors, as described in more detail herein.

Certain aspects of the present disclosure provide artificial neural network (ANN), convolution neural network (CNN), and Graph Neural Networks (GNN) methods and systems for automatically generating curated interventions in response to patient behavior. In various embodiments, graph-structure data, specifically hypergraph, are employed with representative learning and embedding for determining and generating recommendations for one or more optimal curated interventions for modifying patient behaviors in a patient. The hypergraph comprises one or more patient behavior and recommended input-output relationships mapped as nodes, vertices, hyperedges. The hypergraph is embedded into one or more vectors and processed using a framework for unsupervised learning of patient behavior and intervention recommendation comprising one or more auto encoder-decoder frameworks.

Certain aspects of the present disclosure provide methods and a system for determining and generating one or more optimal curated interventions for modifying patient behaviors in a patient. In various embodiments, the system comprises a suite of sensors including but not limited to CCD camera, wearable sensors, passive sensors, Internet of Things (IoT) sensors, or the like. The one or more said sensor enables the recording or observation of a patient's daily living activity and/or behavior. In various embodiments, the system comprises a mobile application (mAPP) executable on a mobile computing platform (e.g., mobile phone). In alternative embodiments, the system comprises a web application (wAPP) executable on a stationary computing platform (e.g., desktop computer). The mobile or stationary computing platform enables a caregiver to register and receive intervention recommendations from remote server using said mAPP or wAPP. The remote server comprises one or more computing system and methods for processing, analyzing sensor or caregiver generated data relating to patient activity or behavior and generates one or more behavioral modification recommendations or actions to a caregiver. In various embodiments, the said computing system receives, processes, and generates one or more output relating a patient's vital, behavior, environmental status, risk of fall, hazards, or the like. In various embodiments, the system generates one or more alert based on one or more configurable threshold relating to a sensor value, a patient vital, a caregiver input, combinations thereof, or the like. An object of the inventive methods and systems of the present disclosure is to enable the reduction in cost of care, including but not limited to, medicine prescription, medical check-up, care episode, emergency service, hospitalization, or the like.

Certain aspects of the present disclosure provide a system architecture for automatically generating curated interventions for modifying patient behaviors in a patient. The system is configured to enable data ingestion, storage, generation of machine learning (ML), analysis, and intervention prediction. In various embodiments, the system architecture comprises one or more inputs, third-party communication channel and output(s). In various embodiments, the one or more inputs may include, but are not limited to, system logs, caregiver inputs from said mAPP or wAPP, sensor and IoT device, camera, combinations thereof, or the like. In various embodiments, one or more third-party communication channel includes, but is not limited to, an Application Programming Interface (API) grid, or the like. In various embodiments, one or more system output includes, but is not limited to, data sent to IoT device App, data presented to an Analytical User Interface (UI), combinations thereof, or the like. In various embodiments, the said architecture is implemented on one or more remote server, cloud-based server or service, cloud computing, on-demand computing, software as a service (SaaS), computing platform, network-accessible platform, data centers, or the like. In various embodiments, the cloud computing platform comprises one or more computing module or database consisting of, but not limited to a(n): Identity and Access Management (IAM) and Secrets Management, Data Factory, Data Lake Storage, Machine Learning (ML) engine, ML library, SQL Data Warehouse, Analysis Service, Business Intelligence (BI), Web Application, combinations thereof, or the like. The architecture enables the communication/reception of one or more input, third party input, and the generation of one or more output of prediction and/or recommendation of behavioral interventions to a caregiver. In various embodiments, the architecture facilitates the ingestion of said inputs, storage, preparation, training of at least one ML model, and serving the model output of one or more prediction, behavorial intervention, or care recommendation to said Analytical UI, IoT/Device app, mAPP, wAPP, or the like.

Further aspects and embodiments of the present disclosure provide for a computer-implemented method for generating a curated medical intervention, comprising providing, with a remote server communicably engaged with a client device, an instance of an end user application to the client device, the instance of the end user application comprising a graphical user interface rendered at a display of the client device, wherein the instance of the end user application is instantiated by an authorized end user of the end user application, the authorized end user comprising a caregiver for a patient under care; receiving, with the client device, one or more user-generated inputs from the authorized end user via the graphical user interface, the one or more user-generated inputs comprising a patient selection input and at least one observed patient behavior input, wherein the patient selection input comprises a patient identifier configured to identify the patient under care within an application database communicably engaged with the remote server; processing, with the remote server or the client device, the one or more user-generated inputs to determine one or more variables associated with the patient selection input and the at least one observed patient behavior input; analyzing, with the remote server, the one or more user-generated inputs according to an ensemble machine learning framework to generate an intervention recommendation for the patient under care, the intervention recommendation comprising an optimal curated intervention selected from a plurality of curated interventions based on an output of the ensemble machine learning framework, wherein the ensemble machine learning framework comprises a neural network configured to analyze an optimum of one or more time, resource and efficacy variables for the plurality of curated interventions to determine the optimal curated intervention; and presenting, with the client device, the intervention recommendation for the patient under care to the authorized end user via the graphical user interface of the instance of the end user application.

Still further aspects and embodiments of the present disclosure provide for a computer-implemented method for generating a curated medical intervention, comprising receiving, with a plurality of sensors communicably engaged with a remote server, a plurality of sensor input data comprising a plurality of patient activity data or patient behavior data for a patient under care, the plurality of sensors comprising one or more of a camera, a physiological sensor, a wearable sensor and an acoustic sensor; processing, with the remote server, the plurality of sensor input data to extract one or more features for the plurality of patient activity data or patient behavior data for the patient under care, wherein the one or more features comprise one or more variables in an ensemble machine learning framework; analyzing, with the remote server, the plurality of patient activity data or patient behavior data according to the ensemble machine learning framework to generate an intervention recommendation for the patient under care, the intervention recommendation comprising an optimal curated intervention selected from a plurality of curated interventions based on an output of the ensemble machine learning framework, wherein the ensemble machine learning framework comprises a neural network configured to analyze an optimum of one or more time, resource and efficacy variables for the plurality of curated interventions to determine the optimal curated intervention; communicating, with the remote server, the intervention recommendation to a client device executing an instance of an end user application; and presenting, with the client device, the intervention recommendation within a graphical user interface of the end user application to an authorized end user, wherein the authorized end user comprises a caregiver for the patient under care.

Still further aspects and embodiments of the present disclosure provide for a computer-implemented method for generating a curated medical intervention, comprising receiving, with a remote server via an end user device, a plurality of patient activity data or patient behavior data for a patient under care, the plurality of patient activity data or patient behavior data comprising one or more of a plurality of user-generated inputs from an authorized end user via a graphical user interface of an end user application and a plurality of sensor inputs from one or more sensors, wherein the authorized end user comprises a caregiver of the patient under care; storing, with an application database communicably engaged with the remote server, the plurality of patient activity data or patient behavior data; processing, with the remote server, the plurality of patient activity data or patient behavior data according an ensemble machine learning framework, wherein the plurality of patient activity data or patient behavior data comprises a training dataset for the ensemble machine learning framework, wherein the training dataset is stored in the application database; analyzing, with the remote server, the plurality of patient activity data or patient behavior data according the ensemble machine learning framework to generate an intervention recommendation for the patient under care, the intervention recommendation comprising an optimal curated intervention selected from a plurality of curated interventions based on an output of the ensemble machine learning framework, wherein the ensemble machine learning framework comprises a neural network configured to analyze an optimum of one or more time, resource and efficacy variables for the plurality of curated interventions to determine the optimal curated intervention; and presenting, with the user device, the intervention recommendation for the patient under care to the authorized end user via the graphical user interface of the end user application.

The foregoing has outlined rather broadly the more pertinent and important features of the present invention so that the detailed description of the invention that follows may be better understood and so that the present contribution to the art can be more fully appreciated. Additional features of the invention will be described hereinafter which form the subject of the claims of the invention. It should be appreciated by those skilled in the art that the conception and the disclosed specific methods and structures may be readily utilized as a basis for modifying or designing other structures for carrying out the same purposes of the present invention. It should be realized by those skilled in the art that such equivalent structures do not depart from the spirit and scope of the invention as set forth in the appended claims.

### BRIEF DESCRIPTION OF DRAWINGS

The above and other objects, features and advantages of the present disclosure will be more apparent from the following detailed description taken in conjunction with the accompanying drawings, in which:

FIG. 1 shows an example of a flow diagram for a system for automatically generating curated interventions in response to patient behavior;

FIG. 2 shows an example of a flow diagram for a method for automatically generating curated interventions in response to patient behavior including generating updated intervention vectors after updated intervention generator training;

FIG. 3 shows an example of a high-level flow diagram for a system for automatically generating curated interventions in response to patient behavior including generating updated intervention vectors after updated intervention generator training;

FIG. 4 shows a more detailed example of an intervention processor as used in a system for automatically generating curated interventions;

FIG. 5 shows one example of an artificial neural network implemented in an intervention processor as used in a system for automatically generating curated interventions;

FIG. 6 schematically illustrates one example of an artificial neural network training technique as may be implemented in an intervention processor used in a system for automatically generating curated interventions;

FIG. 7 is an illustration of the components of a Convolutional Neural Network architecture;

FIG. 8 is a flow diagram for a method for automatically generating curated interventions in response to patient behavior using a hypergraph;

FIG. 9 is an example of the input-output relationship as may be implemented in intervention processor;

FIG. 10 is an example of a graphical autoencoder (GAE) for network embedding;

FIG. 11 is a system for determining and generating one or more optimal curated interventions for modifying patient behaviors in a patient;

FIG. 12 is a system architecture for automated construction, resource provisioning, and execution of machine learning models for generating curated patient behavorial interventions, in accordance with certain aspects of the present disclosure;

FIG. 13 is a process flow diagram of a computer-implemented method for generating a curated medical intervention, in accordance with certain aspects of the present disclosure;

FIG. 14 is a process flow diagram of a computer-implemented method for generating a curated medical intervention, in accordance with certain aspects of the present disclosure; and

FIG. 15 is a process flow diagram of a computer-implemented method for generating a curated medical intervention, in accordance with certain aspects of the present disclosure.

### DETAILED DESCRIPTION

It should be appreciated that all combinations of the concepts discussed in greater detail below (provided such concepts are not mutually inconsistent) are contemplated as being part of the inventive subject matter disclosed herein. It also should be appreciated that terminology explicitly employed herein that also may appear in any disclosure incorporated by reference should be accorded a meaning most consistent with the particular concepts disclosed herein.

It should be appreciated that various concepts introduced above and discussed in greater detail below may be implemented in any of numerous ways, as the disclosed concepts are not limited to any particular manner of implementation. Examples of specific implementations and applications are provided primarily for illustrative purposes. The present disclosure should in no way be limited to the exemplary implementation and techniques illustrated in the drawings and described below.

Where a range of values is provided, it is understood that each intervening value, to the tenth of the unit of the lower limit unless the context clearly dictates otherwise, between the upper and lower limit of that range and any other stated or intervening value in that stated range is encompassed by the invention. The upper and lower limits of these smaller ranges may independently be included in the smaller ranges, and are also encompassed by the invention, subject to any specifically excluded limit in a stated range. Where a stated range includes one or both of the endpoint limits, ranges excluding either or both of those included endpoints are also included in the scope of the invention.

Before the present invention and specific exemplary embodiments of the invention are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, exemplary methods and materials are now described. All publications mentioned herein are incorporated herein by reference to disclose and describe the methods and/or materials in connection with which the publications are cited.

Any publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

Unless the context requires otherwise, throughout the specification and claims which follow, the word “comprise” and variations thereof, such as, “comprises” and “comprising” are to be construed in an open, inclusive sense that is as “including, but not limited to.” Reference throughout this specification to “one example” or “an example embodiment,” “one embodiment,” “an embodiment” or combinations, plural forms, and/or variations of these terms means that a particular feature, structure, or characteristic described in connection with the example is included in at least one embodiment of the present disclosure. Thus, the appearances of the phrases “in one example” or “in an example” in various places throughout this specification are not necessarily all referring to the same embodiment. Furthermore, the particular features, structures, or characteristics may be combined in any suitable manner in one or more embodiments.

An “artificial neural network” (sometimes simply called “neural network”) is a computer software program comprising a series of algorithms that endeavors to recognize underlying relationships in a set of data through a process that mimics the way the human brain operates.

A “deep neural network” (DNN) as used herein means an artificial neural network (ANN) with multiple layers between the input and output layers. Each mathematical manipulation as such is considered a layer.

Bluetooth® technology, as used herein means a commercially available low-power wireless connectivity technology used to stream audio, transfer data and broadcast information between devices. This technology is available from Bluetooth SIG, Inc. of Kirkland, Wash.

As used herein, “cellular telephone” (or “smart phone”) has its generally accepted meaning and includes any portable device that can make and receive telephone calls to and from a public telephone network, which includes other mobiles and fixed-line phones across the world. It also includes mobile devices that support a wide variety of other services such as text messaging, software applications, MMS, e-mail, Internet access, short-range wireless communications (for example, infrared and Bluetooth® technology).

As used herein, “plurality” is understood to mean more than one. For example, a plurality refers to at least two, three, four, five, ten, 25, 50, 75, 100, 1,000, 10,000 or more.

As used herein, the terms “computer”, “processor” and “computer processor” encompass a personal computer, a workstation computer, a tablet computer, a smart phone, a microcontroller, a microprocessor, a field programmable object array (FPOA), a digital signal processor (DSP), an application-specific integrated circuit (ASIC), a field programmable gate array (FPGA), a programmable logic array (PLA), or any other digital processing engine, device or equivalent capable of executing software code including related memory devices, transmission devices, pointing devices, input/output devices, displays and equivalents.

As used herein, the term ROC means a Receiver Operating Curve typically created by created by plotting the true positive rate (TPR) against the false positive rate (FPR) at various threshold settings.

As used herein the term, “AUC” refers to the area under the curve, more particularly the area under a Receiver Operating Curve (ROC).

As used herein the term, “obtaining” is understood herein as manufacturing, purchasing, or otherwise coming into possession of.

As used herein, “tablet computer” has its generally accepted meaning and includes any mobile computer including a complete mobile computer, larger than a mobile phone or personal digital assistant, integrated into a flat touch screen and primarily operated by touching the screen such as, for example, an Apple iPad® tablet computer. As used herein “mobile device” includes smart phones and tablet computers.

As used herein the term, “transmit” and its conjugates means transmission of digital and/or analog signal information by electronic transmission, Wi-Fi, Bluetooth® technology, wireless, wired, or other known transmission technologies including transmission to an Internet web site.

As used herein cloud computing includes on-demand computing, software as a service (SaaS), platform computing, network-accessible platform, cloud services, data centers, or the like. The term “cloud” can include a collection of hardware and software that forms a shared pool of configurable computing resources (e.g., networks, servers, storage, applications, services, etc.), which can be suitably provisioned to provide on-demand self-service, network access, resource pooling, elasticity, and measured service, among other features.

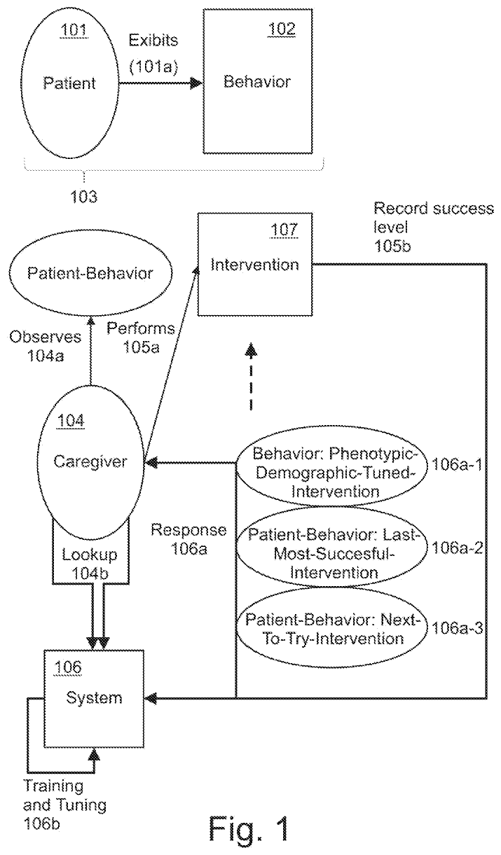

Referring now to FIG. 1, an example of a flow diagram for a system for automatically generating curated interventions in response to patient behavior is shown. In response to inputs and other data as detailed below, a system 106 predicts interventions 107 for behaviors 102 exhibited by patients 101 within particular contexts of care. This particular example envisions a context of patients within a dementia/Alzheimer's unit within a care facility. The common thread in caring for people at home or in care settings is the ability to provide quality care and assist in their activities of daily living (ADLs) with little to no resistance. The invention assists the caregiver in providing specific interventions and information to prevent and reduce aberrant patient behaviors to better care for their needs.

For example, a patient 101 exhibits a behavior 102 which is an observable patient behavior 103. Each such patient behavior 103 includes a set of features which may advantageously be used to build a predictive network. Examples of features include but are not limited to time-of-day, pre/post meal, pre/post-medications, environment bedroom, hallway, bathroom, etc., pre/post activity, prior activity, prior illness, or injury, and/or upcoming activity, prior encounters/outcomes, patient background, combinations thereof, or the like.

In one example, a caregiver 104 observes the patient behavior 103 and uses an application to select the patient 101 and the observed behavior 102. As detailed further below, the system 106 looks up 104a the patient behavior 103, the behavior 102 and the patient 101.

The system 106 records 104a the observed event as an encounter between the caregiver 104, patient 101, behavior 102 and patient behavior 103 and looks up 104b a vector of responses based on a neural network which is constantly trained. In one example, the system 106 returns 106a a vector of responses to the inquiry consisting minimally of responses including, for example: patient behavior last-most-successful-intervention 106a-2, patient behavior next-to-try-intervention 106a-3, and behavior phenotypic-demographic-tuned-intervention 106a-1.

Each response in the vector of responses from 106a is specific in use and how it is processed as detailed here. The patient behavior last-most-successful-intervention 106a-2, returns, for the given patient 101 and behavior 102, the last response which was marked as successful for that given combination. The patient behavior next to try intervention 106a-3, returns, for the given patient 101 and behavior 102, the next calculated intervention 107 for the given behavior for that patient, based on network in the system 106 given the last set of provided vectors. For clarity, if a patient behavior 103 is observed at time n by a caregiver 104 who then uses a given intervention 107, and subsequently marks the intervention 107 as un-successful, then 106a-3 returns the next intervention from that same list. The behavior phenotypic-demographic-tuned-intervention 106a-1 is the default return if there has been no prior record of the behavior 101 for the patient 102 and returns the most efficacious intervention 107 for the given behavior given the known patient demographic and phenotypic data.

The system 106 records each encounter. Each such encounter, between a patient 101, and a caregiver 104 carries the data in patient behavior 103 as described above. This set of features is merged with the behavior features and patient features and the network adds this encounter, along with each Intervention 107-106a-x and the success or failure thereof, to continuously train and improve the network accuracy. In an alternative embodiment, the set of features is merged with the behavior features and patient features and the network adds this encounter, along with each Intervention 107-106a-x and the duration, resources, frequency, intensity, and effectiveness of the intervention thereof. On reading the returned set of responses 106-x, where x represents any letter, the caregiver 104 performs 105a the suggested intervention 107. The caregiver observes 105b the level of success of the intervention and indicates the success level 106b on the mAPP which the system 106 records and ties to the encounter. In an alternative embodiment, the caregiver observes 105b the level of success and indicates the success level 106b combined with a duration, resource, intensity, frequency, and effectiveness of the intervention on the mAPP which the system 106 records and ties to the encounter. Note that the success, effort, duration, frequency, intensity, efficiency value can be any response from binary to a float to a vector.

The system 106 performs continuous training and tuning 106b with each recorded success 105b for each encounter. In this example, this continuous training improves the predictive accuracy [(True-Positive+True-Negative)/Total] across combinations of features, and specifically in this example, within the context of predicting the most accurately efficient intervention 107 based on patient 101 demographic+phenotypic data and behavior 102 data, as collected in patient behavior 103.

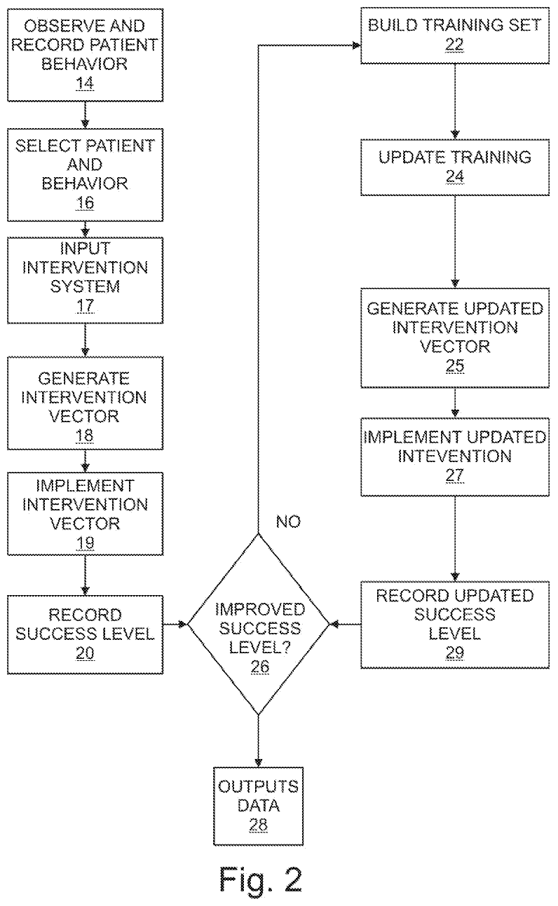

Referring now to FIG. 2, an example of a flow diagram for a method for automatically generating curated interventions in response to patient behavior including generating updated intervention vectors after updated intervention generator training is shown. A computer-implemented method for automatically generating curated interventions for modifying patient behaviors in at least one patient includes observing at least one patient behavior 14, wherein at least one patient behavior includes a set of features. The next process step includes operating a computer processor to execute a program for selecting at least one patient and at least one patient behavior from a table 16. The next process step includes operating the computer processor to input the selected patient and the selected patient behavior into a trained intervention generating system 17. Next, the trained intervention generating system is operated to generate at least one intervention responsive to the selected patient and the selected patient behavior inputs 18. The next process step includes implementing the intervention vector 19. The next process step includes recording a success level for at least one generated intervention 20. In an alternative embodiment, a duration, resource, intensity, frequency, and effectiveness of the intervention 20 is also recorded.

Having carried out an initial set of process steps, the method continues by querying whether an improved success level has been achieved 26. If the success level has not been improved to the satisfaction of, for example, the caregiver, then the method proceeds to the process step of building a training set including selected patient, the selected patient behavior inputs, and the success level 22. In another step, training of the intervention generating system is updated 24 by inputting the training set and allowing the intervention generating system to update its model weights to improve mapping of the inputs to the outputs. After training an updated intervention vector is generated 25. The updated intervention vector is transmitted to the caregiver which implements the intervention and records the level of success 29. The resulting level of success is compared against the desired level 26 and the updating process may be repeated until the desired improved success level is attained. In an alternative embodiment, the updated intervention vector is transmitted to the caregiver which implements the intervention and records the level of success 29 and a corresponding duration, resource, intensity, frequency, and effectiveness of the intervention. The resulting level of success is compared against the desired level 26 and corresponding duration, caregiving resource, intensity, frequency, and effectiveness of the intervention and the updating process may be repeated until the desired improved success level is attained. Once the desired improved level of success is attained outputs data may be provided 28. Alternatively, outputs data may be provided at intermediate steps.

In one example the set of features includes time-of-day, pre-meal and post meal, pre-medications, post-medications, environment, pre/post activity, prior-activity, upcoming-activity, prior-illness or injury, and combinations thereof. In another example, the set of features include behavior symptoms such as aggression, repetition, wandering, pacing, fidgeting, verbal outburst, change in eating behaviors, apathy, hoarding, change in mood, change in personality, and trouble communicating with others.

In another example the program may advantageously reside in a computer workstation, personal computer, remote server, cloud computing platform, or mobile device such a tablet computer, smart phone, or the like.

In another example at least one intervention vector may include categories such as patient behavior last-most-successful-intervention, patient behavior next-to-try-intervention, behavior phenotypic-demographic-tuned-intervention, duration, resource, intensity, frequency, and effectiveness of the intervention, and combinations thereof. In another example, the act of operating the trained intervention generating system comprises operating a trained neural network.

In yet another example, the active updating training of the intervention generating system comprises updating training of a neural network.

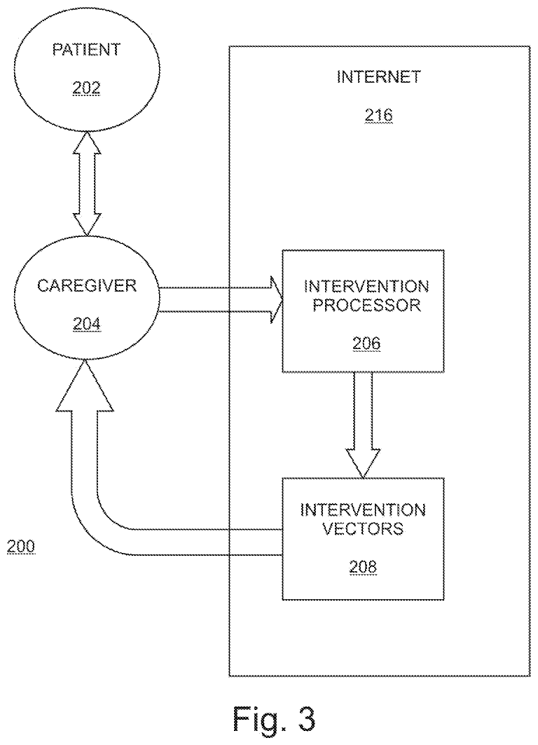

Referring now to FIG. 3, an example of a high-level block diagram for a system for automatically generating curated interventions in response to patient behavior including generating updated intervention vectors after updated intervention generator training is shown. The system 200 includes a patient 202, a caregiver 204 and an intervention processor 206. In a typical example of implementation, a patient 202 exhibits a patient behavior which is observed by the caregiver 204. The caregiver 204 using a mobile device, for example, selects the observed patient behavior and identifies the patient using an application (e.g., mAPP disclosed herein after) loaded into the mobile device. That information is transmitted to the intervention processor 206 which then generates intervention vectors 208. The intervention vectors 208 are transmitted to the caregiver, such as through the caregiver's mobile device. The caregiver would then implement the intervention with the patient and observe the resulting level of success.

In one useful example, the intervention processor 206 and intervention vectors are embedded in and accessed from the Internet 216. As shown below, access to the Internet may be provided by a mobile device which receives transmissions by means of electrically coupled or, preferably, wireless connections such as Wi-Fi, Bluetooth® and the like. In alternative embodiments, access to the Internet by mobile device may be by means of electrically coupled or, preferably, wireless connections, such as 3G, 4G, 4GLTE, GSM, Ethernet, TCP/IP, intranet, local-area network (“LAN”), home-area network (“HAN”), serial connection, parallel connection, wide-area network (“WAN”), Fiber Channel, PCI/PCI-X, AGP, VLbus, PCI Express, Express-card, Infiniband, ACCESS.bus, Wireless LAN, HomePNA, Optical Fiber, G.hn, infrared network, satellite network, microwave network, cellular network, virtual private network (“VPN”), Universal Serial Bus (“USB”), FireWire, Serial ATA, I-Wire, UNI/O, or any form of connecting homogenous, heterogeneous systems and/or groups of systems together.

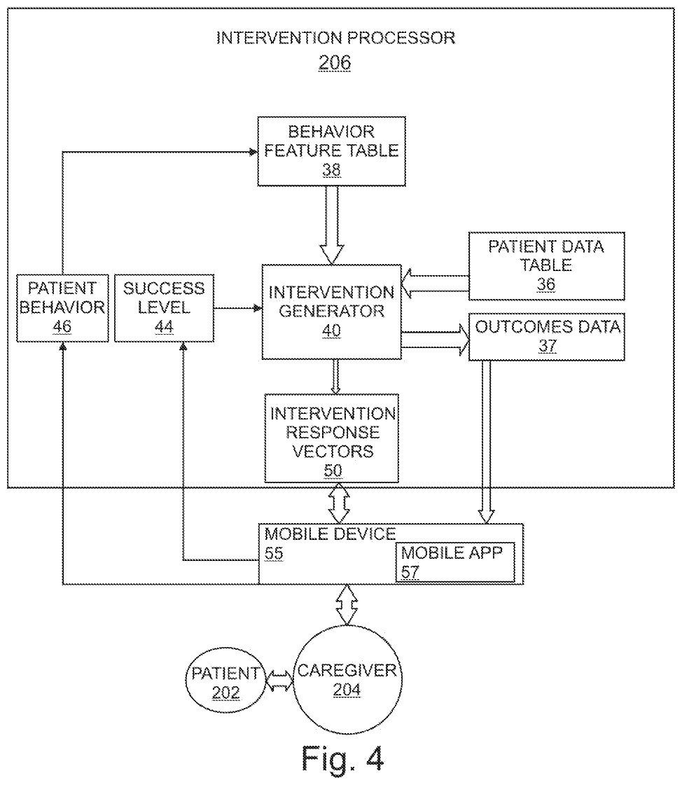

Referring now to FIG. 4, a more detailed example of an intervention processor used in a system for automatically generating curated interventions is shown. An intervention processor 206 includes an intervention generator 40 which generates intervention response vectors 50 in response to inputs. The intervention generator inputs 40 include inputs from a behavior feature table 38 and a patient data table 36. After an initial iteration, inputs may also include success level values 44. In an alternative embodiment, success level values may include one or more corresponding duration, caregiving resource, intensity, frequency, and effectiveness of the intervention. The intervention response vectors 50 are transmitted to a mobile device 55 including a mobile application 57. The mobile device 55 and mobile application 57 are operated by a caregiver 204 who is observing and in communication with the patient 202.

In operation, the caregiver 204 may observe patient behaviors of patient 202 which are then input into the mobile application 57 and communicated to the intervention processor through a patient behavior database 46 and a success level input 44. In an alternative embodiment, a success level input 44 may include a corresponding duration, caregiving resource, intensity, frequency, and effectiveness of the intervention. Outcomes data 37 may be provided by the intervention generator and transmitted to the mobile device 55. Examples of Outcomes data are detailed below.

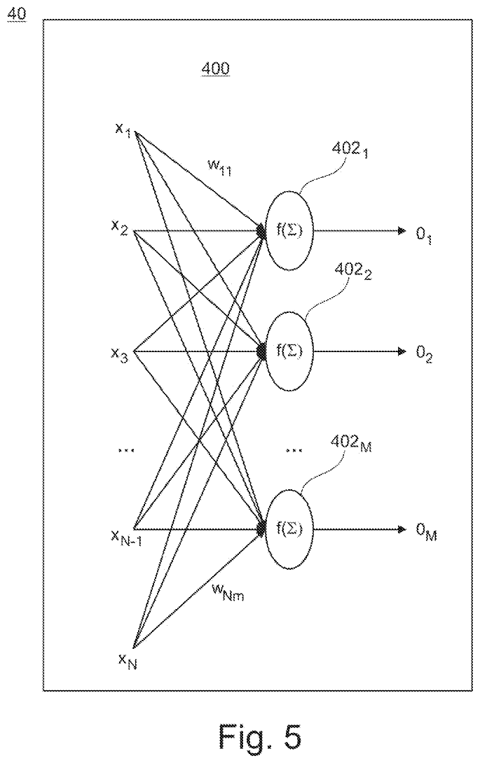

Referring now to FIG. 5, one example of an artificial neural network implemented in an intervention processor as used in a system for automatically generating curated interventions is shown. In one example, the intervention generator 40 may include a neural network 400. A schematic example of a neural network 400 is shown. Inputs include X₁, X₂ . . . Xₙ which may correspond to, for example, behavior features, patient data, success level and any other relevant data. Model weights W₁₁ Wnm are applied to each input node 402₁, 402₂ . . . 402_{M}. Each of the nodes provides an output O₁, O₂ . . . On. The outputs may advantageously comprise an intervention vector which may be transmitted to the caregiver through the mobile application.

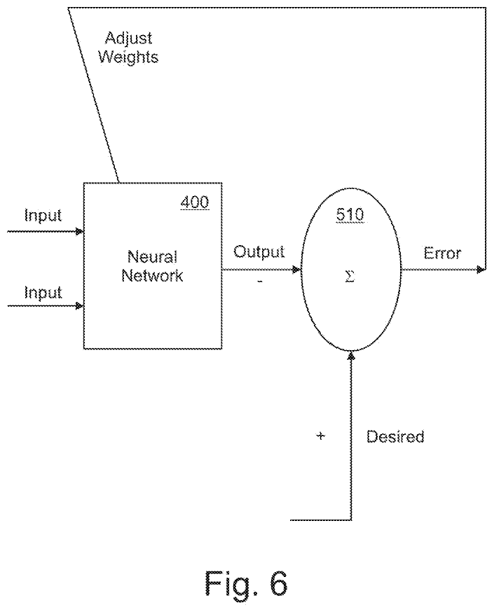

Referring now to FIG. 6, one example of an artificial neural network training technique as may be implemented in an intervention processor used in a system for automatically generating curated interventions is schematically illustrated. During training, neural network 400 receives inputs, as described above, and supplies at least one output which is compared to a desired result in summing junction 510 which produces an error value. The model weights are adjusted in response to the error value reduce the error. In a system for Alzheimer's/dementia patient behavior modification, the desired value may include the level of success value for a selected patient and a selected behavior. In an alternative embodiment, the desired value may include the level of success value and a corresponding duration, caregiving resource, intensity, frequency, and effectiveness of the intervention, or combinations thereof, for a selected patient and a selected behavior.

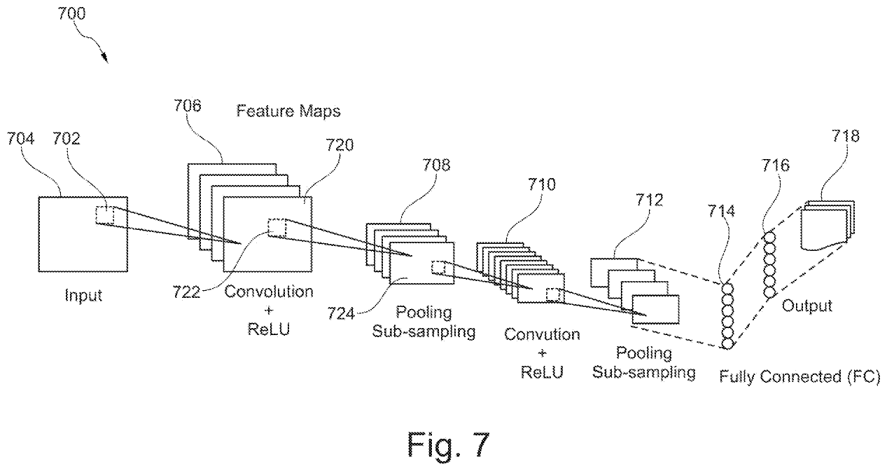

An object of the present disclosure is an intervention generator comprising an artificial neural network (ANN) such as a Convolution Neural Network (CCN) or deep neural network DNN with multiple layers between the input and output layers. FIG. 7 is an illustration of the components of a Convolutional Neural Network (CNN) architecture 700. A CNN is a special type of artificial neural network (ANN). The fundamental difference between a densely connected layer of an ANN (e.g., neural network 400 of FIG. 4) and a convolution layer is that ANNs learn global patterns in their input feature space. In contrast, convolution layers learn local patterns that are usually small 2D windows, patches, filters, or kernels 702 of an input 704. The patterns learned by CNNs are translational invariant, allowing global pattern recognition for generating intervention recommendations. A CNN can also learn spatial hierarchies of patterns whereby a first convolution layer 706 can learn small local patterns such as edges and additional or subsequent layers will learn larger patterns comprising features of the previous or first layer.

In accordance with certain aspects of the present disclosure, CNN architecture 700 learns highly non-linear mappings by interconnecting layers of artificial neurons arranged in many different layers with non-linear activation functions. CNN architecture 700 may comprise one or more convolutional layers 706,710 interspersed with one or more sub-sampling layers 708,712 or non-linear layers, which are typically followed by one or more fully connected layers 744,716. Each element of CNN architecture 700 may receive inputs from a set of features (e.g., patient behaviors, symptoms, etc.) in the previous layer. CNN architecture 700 learns concurrently because the neurons in the same feature map 720 have identical weights or parameters. These local shared weights reduce the complexity of the network such that when multi-dimensional input data enters the network, CNN architecture 700 reduces the complexity of data reconstruction in the feature extraction and regression or classification process.

In accordance with certain aspects of the present disclosure, a tensor is a geometric object that maps in a multi-linear manner geometric vectors, scalars, and other tensors to a resulting tensor. Convolutions operate over 3D tensors (e.g., vectors), called feature maps (e.g., 720), with two spatial axes (height and width) as well as a depth axis (also called the channels axis). The convolution operation extracts patches 722 from its input feature map and applies the same transformation to all of these patches, producing an output feature map 724. This output feature map is still a 3D tensor; having a width and a height. Filters encode specific aspects of the input data at a height level. A single filter could be encoded with, for example, a patient behavior 103 of FIG. 1, including a set of features that may advantageously be used to build a predictive network. Examples of features may include, but are not limited to, time-of-day, pre/post meal, pre/post-medications, environment bedroom, hallway, bathroom, etc., pre/post activity, prior activity, prior illness, or injury, and/or upcoming activity, prior encounters/outcomes, patient background, patient symptoms combinations thereof, or the like.

A convolution operates by sliding these windows of size 3×3 or 5×5 over a 2D or 3D input feature map, stopping at every location, and extracting a patch 722 of surrounding features [shape (window Height, window Width, input Depth)]. Each such patch may then be transformed (via a tensor product with the same learned weight matrix, called the convolution kernel) into an ID vector of shape (output_depth). All of the vectors are then spatially reassembled into, for example, a 3D output map of shape (Height, Width, output Depth). Every spatial location in the output feature map corresponds to the same location in the input feature map (for example, the lower-right corner of the output contains information about the lower-right corner of the input).

During training, certain aspects of CNN architecture 700 may be adjusted or trained so that the input data leads to a specific output estimate. CNN architecture 700 may be adjusted using back propagation based on a comparison of the output estimate and the ground truth (i.e., true label) until the output estimate progressively matches or approaches the ground truth. CNN architecture 700 may be trained by adjusting the weights (w) or parameters between the neurons based on the difference between the ground truth and the actual output. The weights between neurons are free parameters that capture a model's representation of the data and are learned from input/output samples. The goal of model training is to find parameters (w) that minimize an objective loss function L(w), which measures the fit between the predictions the model parameterized by w and the actual observations or the true label (e.g., patient behaviors). In one embodiment, the loss functions are the cross-entropy for classification and mean-squared error for regression. In other implementations, CNN architecture 700 utilizes loss functions such as Euclidean loss and softmax loss.

It is an object of the present disclosure whereby the CNNs are trained with stochastic gradient descent (SGD) using mini-batches. SDG an iterative method for optimizing a differentiable objective function (e.g., loss function), a stochastic approximation of gradient descent optimization. In various embodiments, one or more variants of SGD are used to accelerate learning. These may include AdaGrad, AdaDelta, or RMSprop to tune a learning rate adaptively for each patient behavorial feature. In an alternative embodiment, momentum methods, SGD variants, used to train neural networks. These methods add to each update a decaying sum of the previous updates. In other implementations, the gradient is calculated using only selected data pairs fed to a Nesterov's accelerated gradient and an adaptive gradient to inject computation efficiency.

The convolution layers (e.g.,706,708) of a CNN serve as feature extractors. Convolution layers act as adaptive feature extractors capable of learning and decomposing the input data into hierarchical features. In one embodiment, the convolution layers take a 2D array of patient behavorial features as input and produce a third array as output. In such an implementation, convolution operates on 2D data, with one array being the input array 704 and the other array, the kernel (e.g.,702), applied as a filter on the input array 704, producing an output array. The convolution operation includes sliding the kernel 702 over the input array 704. For each position of the kernel 702, the overlapping values of the kernel and the input array 704 are multiplied and the results are added. The sum of products is the value of the output array 720 at the point in the input array 704 where the kernel 702 is centered. The resulting different outputs from many kernels are called feature maps (e.g., 720,724).

Once the convolutional layers (e.g., 706, 710) are trained, they are applied to perform recognition tasks on new inference data. Convolution layers use convolution filter kernel weights, which are determined and updated as part of the training process. The convolution layers extract different features of the input 704, which are combined at higher layers (e.g.,708,710,712). In various embodiments, said CNN uses a various number of convolution layers, each with different convolving parameters such as kernel size, strides, padding, number of feature maps, and weights.

It is an object of the present disclosure to employ sub-sampling layers (e.g., 708, 712) to reduce the resolution of the features extracted by the convolution layers to make the extracted features or feature maps (e.g., 720,724) robust against noise and distortion, reduce the computational complexity, to introduce invariance properties, and to reduce the chances of overfitting. In one embodiment, sub-sampling layers (e.g., 708,712) employ two types of pooling operations; average pooling and max pooling. The pooling operations divide the input into non-overlapping two-dimensional spaces. For average pooling, the average of the four values in the region is calculated for pooling. The output of the pooling neuron is the average value of the input values that reside with the input neuron set. For max pooling, the maximum value of the four values is selected for pooling. Max pooling identifies the most predictive feature within a sampled region and reduces the resolution and memory requirements of the image.

It is another object of the present disclosure to employ one or more non-linear layer within a CNN for neuron activation in conjunction with convolution. Non-linear layers use different non-linear trigger functions to signal distinct identification of likely features on each hidden layer (e.g., 706,710). In various embodiments, non-linear layers use a variety of specific functions to implement the non-linear triggering, including but not limited to the Rectified Linear Unit (ReLU), PreLU, hyperbolic tangent, absolute of hyperbolic tangent, sigmoid and continuous trigger (non-linear) functions. In a preferred implementation, one or more ReLUs are used for activation. ReLU is a non-continuous, non-saturating activation function that is linear with respect to the input if the input values are larger than zero and zero otherwise. In other implementations, the non-linear layer uses a power unit activation function.

It is another object of the present disclosure to employ one or more fully connected (FC) layers 714,716 within a CNN. In various embodiments, these FC layers are used to concatenate the multi-dimension feature maps (e.g., 720,724, etc.) and to make the feature map into a fixed-size category and generating a feature vector for a classification or recommendation output layer 718. In one implementation, global average pooling is used to reduce the number of parameters and optionally replace one or more FC layers for classification, by taking the spatial average of features in the last layer for scoring. In one embodiment, global average pooling generates the average value from each last layer feature map as the confidence factor for scoring, feeding directly into a softmax layer, which maps, for example, n-dimensional data inputs into [0,1]. This allows for interpreting one or more output 718 as probabilities and selection of 2D data inputs with the highest probability.

It is another object of the present disclosure to employ an ensemble of ANNs that are trained continuously with voting outcomes of patient behavorial interventions. In various embodiments, the continuous training and validation in an ensemble will identify optimal parameters or outcomes by patient behavior and phenotype. As an ensemble, additional useful neural networks, for example, include a feedforward neural network, an artificial neuron, a radial basis function neural network, a multilayer perceptron, a convolutional neural network (CNN), a recurrent neural network (RNN), a modular neural network, combinations thereof, and the like.

An ontology is a formal representation of a set of concepts (e.g., patient behaviors) within a domain (e.g., caregiving interventions) and the relationships between those concepts. In accordance with certain aspects of the present disclosure, a system and method for generating a curated medical intervention may comprise an ontology derived, at least in part, from:
raw data including exhibited behaviors, relationship of behaviors to phenotypes, encounters of patients exhibiting behaviors with caregivers, care giver effort, care efficiency metric, patient symptoms, interventions performed by caregivers, recorded outcomes of interventions, duration and effort of intervention, intensity and frequency, inferential differentials of efficaciousness and effort of interventions;
expert curated interventions based on known psychology by behavior, patient symptoms, and
AI-learned data that may be ascertained by training across population groups and subgroups to improve existing interventions (improvements across outcomes, efficacy, effort, cost), and identify new interventions and/or combinations of interventions.

As contemplated herein, a system and method for generating a curated medical intervention may be implemented by collecting data across a broad or local population segment, group or cohort. The system and method for generating a curated medical intervention may advantageously be populated with expert curated known psychology for the given expected population needs and models are built which continuously improve across a ROC until a given measure of AUC is met. Further, data populating the system may be continually collected over time, using expertly curated data, to create models which can be consumed by AI to replicate the efforts of persons within the non-clinical and possibly clinical settings.

The result of implementing the system and method for generating a curated medical intervention in the above-described manner defines an ontology which will underpin models enabling reliable and predictable healthcare modification of patient behavior. The system and method for generating a curated medical intervention may be implemented in a platform technology, preferably a cloud computing platform, and accompanying mobile application that supports value-based caregiving and outcomes data. Certain illustrative benefits of the present system and method for generating a curated medical intervention include:
a) Caregivers/nurses must record patients' behaviors in real-time rather than at end of shift or when time permits. This action by the caregiver/nurses autogenerates patient behavior plans.
b) Based on data records, caregivers receive suggested interventions on how to calm and de-escalate patient behaviors if/when they become agitated.
c) Shift-changes will be more productive, sharing efficiently what has transpired and what intervention has helped with each patient over the past few hours, days, weeks, or specified timeframe.
d) Real-time, automated documented services can be generated, allowing owners/Executive Directors to justify/bill for additional services and appropriately schedule staff
e) Caregiving resource management
f) Training can be delivered integrated into the workflow rather than taking staff off-line for 4-6 hours.
g) Outcomes data can show, for example:
   i. decrease in psychotropic medications (many anti-psychotics have Black-Box warnings and side effects such as sedation increase the risk of falls);
   ii. decrease in hospitalizations based on behaviors (long term care facility patients have a higher risk for hospitalizations due to medical issues);
   iii. identifiable trends in behaviors for further training and/or deeper understanding of possible environmental factors;
   iv. level or effort of care required for a specific behavorial intervention; and
   v. duration, caregiving resource, intensity, frequency, and effectiveness of the intervention

As described in detail herein, embodiments of the present disclosure provide for an ontology around a descriptive (e.g., a diagnosis) to event (e.g., an encounter) to intervention (e.g., a treatment) to treatment-provider (e.g., a caregiver) to temporal-state (e.g., a season, time-of-day, trigger event). Further, the ontology will be able to describe such behaviors across variable time periods. For example, a given patient may exhibit particular behavior patterns over a period of months, waxing and waning, with assorted interventions, each of which may result in differing outcomes and yet the underlying behavior pattern is the same over the entire period.

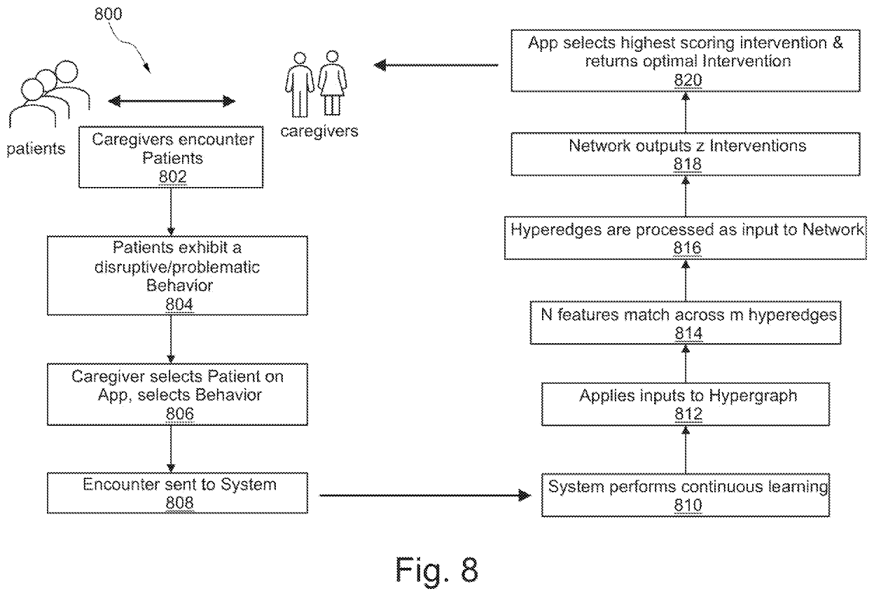

It is an object of the present disclosure to employ an artificial neural network (ANN) method that utilizes graph-structured data for implementation in an intervention processor for automatically generating curated interventions. The graph-structure data comprises one or more generalized data structure for relation modeling. In various embodiments, the graph-structure data is a hypergraph composed of a vertex or node set and a hyperedge set, whereby a hyperedge contains a flexible number of vertices (nodes). Edges (or nodes) in a hypergraph contain features of patients or patient behaviors. In various embodiments, hyperedges are used to model one or more non-pair-wise relations between an observed patient behavior and a recommended behavioral intervention. Referring now to FIG. 8, an example of a flow diagram for a method 800 for automatically generating curated interventions in response to patient behavior using a hypergraph is shown. Method 800 may comprise one or more caregiver (e.g., caregiver 204 of FIG. 4) encounter step 802 with a patient (e.g., 202 of FIG. 4) whereby said patient exhibits 804 a disruptive/problematic behavior. Caregiver 204 may then use mobile application 57 of FIG. 4 to select 806 one or more observed behaviors from said app. The encounter is sent 808 to the intervention process 206 of FIG. 4 preferably via one or more said communication channels to a cloud computing system. In various embodiments, the intervention processor 206 of FIG. 4 performs 810 continuous learning using the one or more input behaviors selected by said caregiver. The intervention processor 206 applies 812 the inputs into a hypergraph whereby N patient behavioral features are matched 814 across m hyperedges. These hyperedges are processed 816 as input to a network (e.g., an ANN as disclosed herein). One or more network (e.g., 400 of FIG. 5, CNN of FIG. 7) is used to generate 818 one or more z-Interventions. In final step, mobile application 57 of FIG. 4 selects the highest scoring intervention and returns 820 the optimal intervention to said caregiver 204 of FIG. 4. In various embodiments, the said method is executed in one or more continuous artificial intelligent system disclosed herein and initially trained with a sufficient of data. In one embodiment, data is added on a continuous ongoing basis. In various embodiments, one or more patient behavior feature data are added as one or more nodes on hyperedges. In one embodiment, each patient encounter 802 includes data about said patient and these nodes are matched across one or more hyperedges. In various embodiments, hyperedges are processed by intervention processor 206 of FIG. 4 as vector inputs to the said network. The network is retrained using new data continuously. One or more output interventions are weighted with the score of matching nodes to the input hyperedges through one or more input-output relationship.

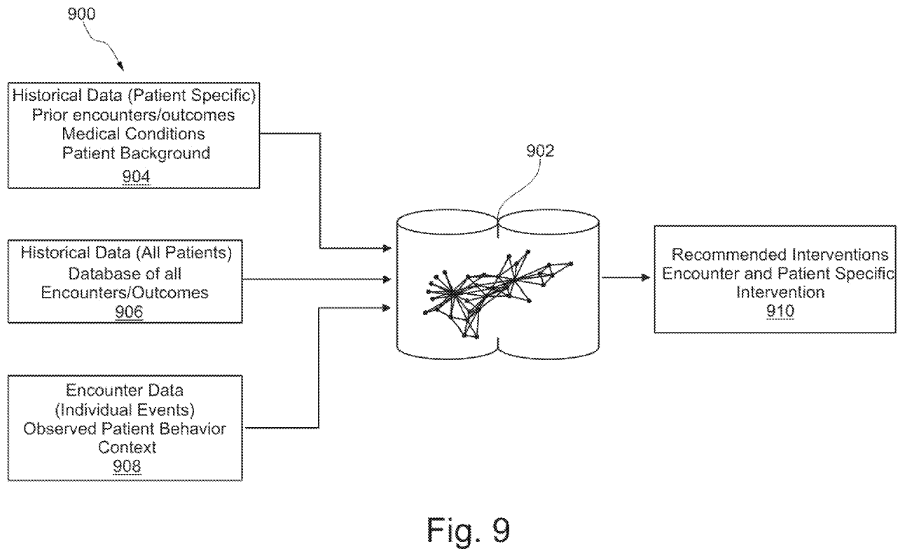

Referring now to FIG. 9, one example of the input-output relationship as may be implemented in intervention processor 206 of FIG. 4, used in a system 902 for automatically generating curated interventions is schematically illustrated. Intervention processor 206 (shown in FIG. 4) with system 902 receives one or more Historical Data 904 that are patient specific; Historical Data 906 from a patient population; or an Encounter Data 908 from individual events between a patient and a caregiver. In various embodiments, Historical Data 904 comprises, for example, one or more prior encounters/outcome, medical conditions, patient background, or the like patient specific data. In various embodiments, Historical Data 906 comprises, for example, one or more data from all encounters/outcomes of a specific or general population of patients. In various embodiments, Encounter Data 908 comprises, for example, one or more individual events, including but not limited to an: observed patient behavior; context; environment; caregiver background; caregiver to patient relationship; completed intervention; completed outcome; caregiver level of effort for an intervention; care efficiency metric for an intervention; duration, frequency, intensity, and effectiveness of an intervention. In one embodiment, Historical Data 904 and 906 may be stored in patient data table 36 of FIG. 4. In another embodiment Encounter Data 908 may be stored in table 37 of FIG. 4. In various embodiments, inputs 904,906,908 are processed by intervention generator 40 of FIG. 4 using one or more said trained ANN (e.g., an ANN of FIGS. 5 & 7) to generate an output 910. The output 910 is a recommended intervention that includes, but not limited to, an encounter and patient specific intervention. In various embodiments, a specific intervention may focus on observed or recorded behavior inputs and modifying triggers or activators of the behavior. An intervention may be based on one or more psychological principles or based on careful and systematic description, observation, or record of behaviors—including their timing, frequency, and severity—as well as the environmental circumstances before, during, and after the behavioral symptoms. For example, dementia-related behavorial are often triggered by one or more factors, including but not limited to: the presence of an unmet physical need: such as hunger, pain, or fatigue; environmental conditions, such as overstimulation or under stimulation; or difficulties interpreting verbal, visual, or tactile cues. In this case, output 910 may be provided to caregiver 204 of FIG. 4 that recommends the removal or avoidance of the trigger with an adequate description the target behavior intervention and to isolate the triggers. In various embodiments, caregiver 204 of FIG. 4 uses mobile application 57 of FIG. 4 to record one more patient observations, duration, frequency, intensity, or effectiveness of the target intervention.

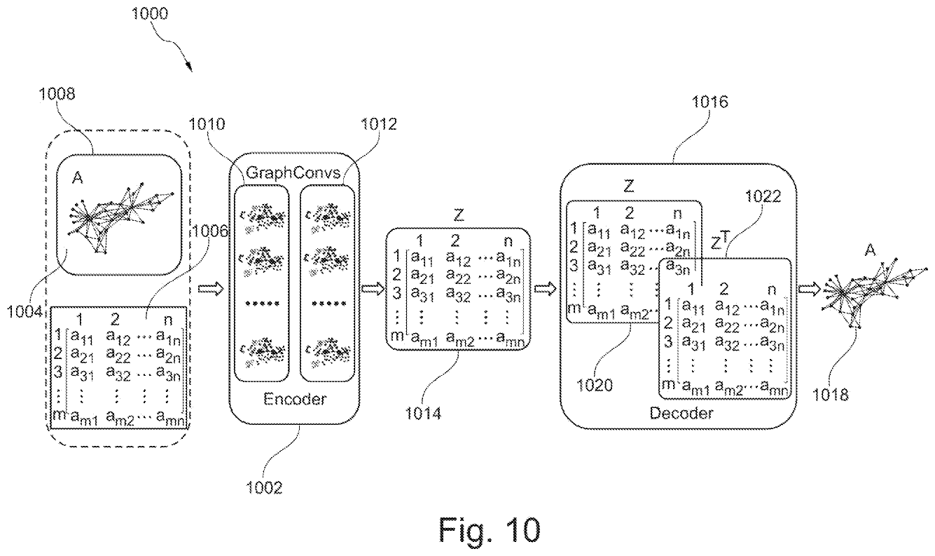

It is an object of the present disclosure to employ an artificial neural network (ANN) system in combination with the method of FIG. 8 that utilizes graph-structured data for implementation in an intervention processor for automatically generating curated interventions. The ANN comprises one or more Graph Neural Networks (GNN) operating on one or more graphs comprising one or more said nodes and hyperedges, for example, connections between one or more said input-output relations of FIG. 9. GGN is employed to iteratively aggregate patient behavior feature information and local graph neighborhoods using ANNs. In various embodiments, one or more network embedding or representation of at least one patient behavior or intervention into at least one low dimension vector and used to generate an intervention recommendation based on but not limited to similarity, strength, statistical properties, node degree, number of hyperedges, clustering coefficient, neighborhood overlap, or the like between nodes and edges. In alternative embodiments, one or more convolutions operations (e.g., described by FIG. 7) transforms and aggregate feature information from a node's one-hop graph neighborhood and by stacking multiple said convolutions information can be propagated across a graph and leverages patient behavior information, as well as their relations to varying interventions. In one embodiment, a framework for unsupervised learning of patient behavior and intervention recommendation on graph-structured data is based on one or more auto encoder-decoder. The model comprises the use of one or more latent patient behavior variables and continuously learns interpretable latent representations for a said hypergraph. Referring now to FIG. 10, an example of a graphical autoencoder (GAE) 1000 for network embedding is show. Graph autoencoders (GAEs) are deep neural architectures which map nodes (e.g., patient behavior) into a latent feature space and decode graph information from latent representations. In various embodiments, a GAE is used to learn network embeddings or generate new graphs. In various embodiments, GAE 1000 learns network embedding using an encoder 1002 to enforce embeddings to preserve a hypergraph 1004. GAE 1000 may comprise topological information using one or more positive pointwise mutual information (PPMI) matrix 1006 and an adjacency matrix A 1008. A representation of hypergraph 1004 may comprise a patient behavior-interaction comprising patient behavior and intervention nodes and hyperedges. In various embodiments, the normalized adjacency matrix A 1008 and the PPMI matrix 1006 captures nodes co-occurrence information through random walks sampled from hypergraph 1004. In various embodiments, GAE 1000 learns the graph embedding in an unsupervised or semi-supervised way in an end-to-end framework or an ensemble of GAEs, exploiting hyperedge level information. In one embodiment, an encoder 1002 employs one or more graph convolution layers 1010, 1012 to embed said hypergraph 1004 into a latent representation Z 1014 upon which an inner product decoder 1016 is used to reconstruct the graph structure. In various embodiments, convolution layers 1010, 1012 comprises one or more elements, components, or a complete CNN network of FIG. 7. In one implementation, an end-to-end framework is built by stacking a several graph convolutional layers followed by a softmax layer for multi-class classification. In another implementation, encoder 1002 consists of graph convolution layer 1010 and graph convolution layer 1012, incorporating a non-linear activation function (e.g., ReLU) to form Z matrix 1014 denoting the network embedding matrix of hypergraph 1004. Decoder 1016 decodes one or more node relational information of hypergraph 1004 from their embedding by reconstructing the graph adjacency matrix Ă 1018. In one embodiment, the decoder 1016 computes a pair-wise distance given the network embeddings. In another embodiment, the decoder 1016 reconstructs or generates the graph adjacency matrix A 1018 the inner product of latent variable matrix Z 1020 and its transposed matrix Z^{T} 1022. In various embodiments, the network may be trained by minimizing the discrepancy between the real adjacent matrix A 1008 and the reconstructed adjacent matrix 1018. In alternative embodiments, the network is trained by minimizing the negative entropy between said matrices 1008 and 1018. In various embodiments, the network is implemented with system 902 of FIG. 9 as a graph-based behavior intervention recommender leveraging the relations between nodes and edges to predict one or more missing link, connection strength, or neighborhood between an observed patient behavior and one or more intervention.

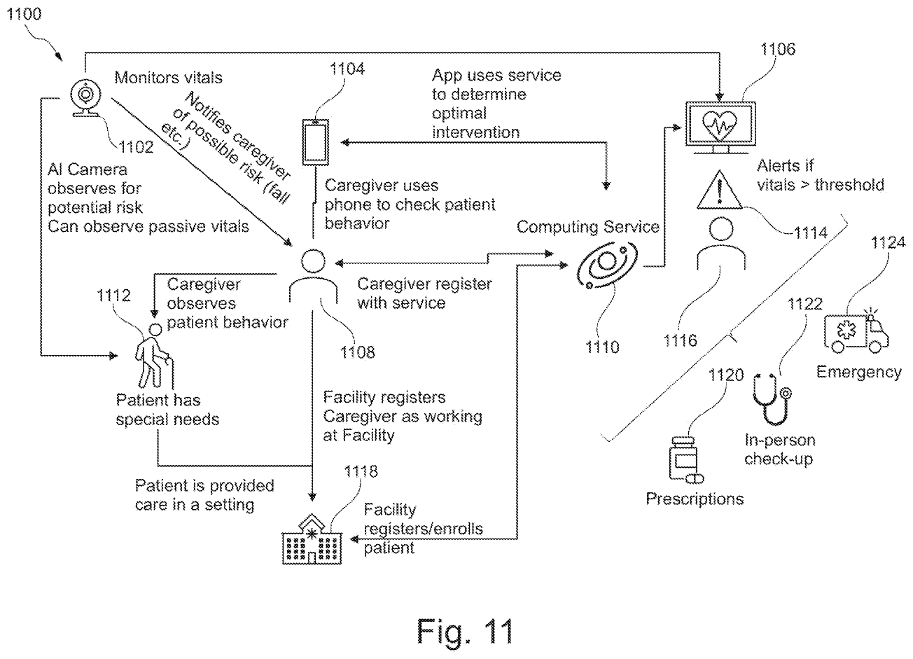

Referring now to FIG. 11, a system 1100 for determining and generating one or more optimal curated interventions for modifying patient behaviors in a patient is shown. The system comprises a suite of sensors including, but not limited to, CCD camera 1102, wearable sensors, passive sensors, Internet of Things (IoT) sensors and the like. The suite of sensors may be configured to enable the recording of patient vitals or observation of a patient's daily living activity or a behavior. In various embodiments, camera 1102 is an AI camera that enables predictions of potential risks of patients falling and the system notifies a staff member or caregiver. In various embodiments, the system comprises a mobile application (mAPP) (e.g., mobile application 57 of FIG. 4) executable on a mobile computing platform (e.g., mobile phone) 1104. In alternative embodiments, system 1100 comprises a web application (wAPP) executable on a stationary computing platform (e.g., desktop computer) 1106. The mobile or stationary computing platform enables a caregiver 1108 to register and receive intervention recommendations from remote computing service 1110 using said mAPP or wAPP while observing a patient 1112. The computing service 1110 comprises one or more computing system and methods for processing, analyzing sensor or caregiver generated data relating to patient 1112 activity or behavior and generates one or more behavioral modification recommendations or actions to caregiver 1108. In various embodiments, the mAPP or wAPP uses computing service 1110 to determine an optimal intervention. In various embodiments, caregiver 1108 uses mobile phone 1104 to check patient 1112 behaviors. In various embodiments, the said computing service 1110 receives, processes, and generates one or more output relating a patient's vital, behavior, environmental status, risk of fall, hazards, or the like. In various embodiments, the system generates one or more alert 1114 based on one or more configurable threshold relating to a sensor value, a patient vital, a caregiver input, combinations thereof, or the like. One or more sensor thresholds used to trigger alert 1114 are configurable by a staff member 1116 of a care facility 1118 whereby data communication methods are managed by computing service 1110. Care facility 1118 can register caregiver 1108 as a working at its location as well as register/enrolls patient 1112 via computing service 1110. In various embodiments, the registration and enrollment steps can be performed using one or more computing platform 1106. In alternative embodiments, the registration and enrollment steps can be performed using one or more mobile device 1104. The methods and system enable the reduction in the cost of care, including, but not limited to, medicine prescriptions 1120, medical check-up 1112, care episode, emergency service 1124, hospitalization and the like.

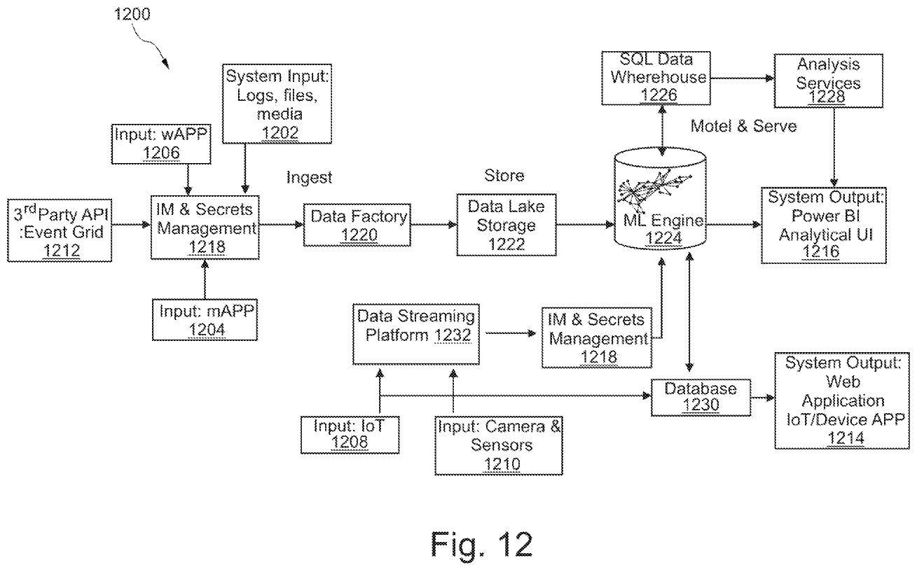

Referring now to FIG. 12, a system architecture 1200 for automated construction, resource provisioning, and execution of machine learning models for generating curated patient behavorial interventions is shown. In various embodiments, system architecture 1200 comprises one or more inputs, third party communication channel, and output. In various embodiments, the one or more input includes, but is not limited to, system logs 1202, caregiver inputs from said mAPP 1204 or wAPP 1206, Internet of Things (IoT) device 1208, Camera & Sensors 1210, combinations thereof, or the like. In various embodiments, one or more third party communication channel includes, but is not limited to, an Application Programming Interface (API) grid 1212, or the like. In various embodiments, one or more system output includes, but is not limited to, data sent to IoT device App 1214, data presented to an Analytical User Interface (UI) 1216, combinations thereof, or the like. In various embodiments, the said architecture is implemented on one or more remote server, cloud-based server or service, cloud computing, on-demand computing, software as a service (SaaS), computing platform, network-accessible platform, data centers, or the like. In various embodiments, the cloud computing platform comprises one or more computing module or database consisting of, but not limited to: Identity and Access Management (IAM) and Secrets Management 1218, Data Factory 1220, Data Lake Storage 1222, Machine Learning (ML) engine 1224, containing an ML library, SQL Data Warehouse 1226, Analysis Service 1228, Database 1230, Business Intelligence (BI) accessible from UI 1216, Web Application, combinations thereof, or the like. System architecture 1200 may enable the communication-reception of one or more input, third party input, and the generation of one or more output of prediction and/or recommendation of behavioral interventions to a caregiver. In various embodiments, the architecture facilitates the ingestion of said inputs, storage, preparation, training of at least one ML model, and serving the model output of one or more prediction, behavorial intervention, or care recommendation to said Analytical UI 1216, IoT/Device app 1214, mAPP (e.g., mobile application 57 of FIG. 4), wAPP, or the like. In accordance with various embodiments, the one more said module or database is implemented using the Azure (Microsoft, Redmond WA) cloud computing platform. In various embodiments, IAM and Secrets Management module 1218 receives one more data transport from streaming platform 1232 capable of processing continuous data from IoT 1208 and/or Camera & Sensors 1210. In one embodiment streaming platform 1232 may comprise AZURE HDINSIGHT (Microsoft, Redmond WA), a cloud distribution of Hadoop® (Apache Software Foundation, Wakefield, Mass.) technology. In various embodiments, streaming platform 1232 enables one or more associated functions such as IoT data extract, transform, and load (ETL). In an alternative embodiment, streaming platform 1232 comprises one or more a real-time streaming data pipeline, message broker for one or more data streams inputs generated by IoT 1208 and Camera & Sensors 1120. In yet another embodiment,

In various embodiments, IAM and Secrets Management module 1218 contains a configurable restricted registration process for managing mAPP and device access associated with a specific care facility. The registration process comprises a first step for downloading said mAPP (e.g., mobile application 57 of FIG. 4). In a second step, a caregiver (e.g., 1108 of FIG. 11) or user is prompt to answer one or more credential questions or inquiries. On completion of answering questions, said mAPP registers answers with cloud computing service 1110 of FIG. 11. The service then generates a 6 character (capital letter+0 through 9) “appcode” being unique to the installation. In a third step, the mAPP registers the responses with said remote cloud computing service 1110 of FIG. 11. In various embodiments, cloud computing service 1110 generates a unique “appcode” that is unique to a specific user installation. In one embodiment, a caregiver or user visits care facility 1118 (as shown in FIG. 11) and provides an appcode to a facility manager who then enters the code into said cloud computing service 1110 of FIG. 11 using an online web portal accessible via desktop computing 1106 (as shown in FIG. 11). In an alternative embodiment, a care facility manager 1116 of FIG. 11 can guide a caregiver or user to download said mAPP and then call or text an appcode to the said manager for registration using said online portal. In various embodiments, said appcode is then related to a specific said mobile or stationary platform and/or user which can be added/deleted/suspended/or removed at a per-unit basis.

In certain further aspects and exemplary embodiments, architecture 1200 is configured for input data ingestion, storage, ML model generation, ML training, data analysis, and prediction of patient interventions via one or more ML pipelines. In various embodiments, Data Factory 1220 enables data integration and data transformation and subsequent storage in Data Lake Storage 1222. In one embodiment, data is ingested and transferred as real-time continuous input into one or more ML model of ML engine 1224 disclosed here, for training, processing, and generating one or more intervention predictions or recommendations. One or more model outputs may be stored in SQL Data Warehouse 1226 and subsequently analyzed using one or more Analysis Services 1228. In various embodiments, architecture 1200 enables the execution the one or more software module to coordinate pipeline elements, processes, and functions by configuring specifications, allocating, elastic provisioning-deprovisioning execution of computing resources, and the control of task transports to and from external inputs and system outputs. In various embodiments, a ML pipeline may be configured to using AZURE DATABRICKS (Microsoft, Redmond WA) and ML engine 1224 to perform data science experimentation, exploration, or analysis to create an end-to-end AI or ML model lifecycle process for generating and serving patient interventions analytics via Analytical UI 1216. In another embodiment, ML engine 1224 functions together with database 1230 for generating and serving patient intervention recommendations to caregivers or facility staff via IoT/Device App 1214. In one embodiment, database 1230 comprises a multi-model database service, for example, AZURE COSMOS DB (Microsoft, Redmond Wash.), leveraging one or more software containers, a standard unit of software that packages code instructions and all dependencies for reliable and fast execution on independent computing environments. In various embodiments, architecture 1200 enable real-time, live pipeline execution, ensuring that ML engine 1224 accept streaming data from inputs (e.g., 1208 & 1210), accept and service request in real-time for generating patient interventions.

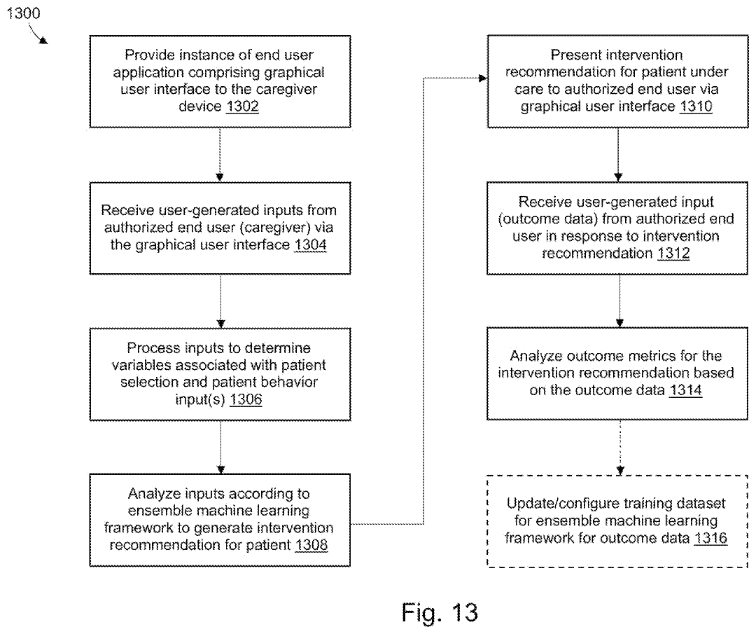

Referring now to FIG. 13, a process flow diagram of a computer-implemented method 1300 for generating a curated medical intervention is shown. Method 1300 may comprise one or more of process steps 1302-1316. In accordance with certain aspects of the present disclosure, method 1300 may be implemented, in whole or in part, within one or more routines of a system for generating a curated medical intervention and/or one or more operations of a computer program product for generating a curated medical intervention, as described herein.

In accordance with certain aspects of the present disclosure, method 1300 may be initiated by performing one or more steps or operations for providing (e.g., with a remote server) an instance of an end user application to a client device (Step 1302). In certain embodiments, the client device may be communicably engaged with a remote server comprising instructions stored thereon for a computer program product for generating a curated medical intervention. In certain embodiments, the instance of the end user application comprises a graphical user interface being rendered at a display of the client device. In certain embodiments, the instance of the end user application may be instantiated by an authorized end user of the end user application. The authorized end user may include a caregiver for a patient under care. Method 1300 may proceed by performing one or more steps or operations for receiving (e.g., with the client device) one or more user-generated inputs from the authorized end user via the graphical user interface (Step 1304). In certain embodiments, the one or more user-generated inputs comprise a patient selection input and at least one observed patient behavior input. The patient selection input may include a patient identifier configured to identify the patient under care within an application database communicably engaged with the remote server. Method 1300 may proceed by performing one or more steps or operations for processing (e.g., with the remote server or the client device) the one or more user-generated inputs to determine one or more variables associated with the patient selection input and the at least one observed patient behavior input (Step 1306). Method 1300 may proceed by performing one or more steps or operations for analyzing (e.g., with the remote server) the one or more user-generated inputs according to an ensemble machine learning framework to generate an intervention recommendation for the patient under care (Step 1308). In accordance with certain aspects of the present disclosure, the intervention recommendation may comprise an optimal curated intervention selected from a plurality of curated interventions based on an output of the ensemble machine learning framework. In certain embodiments, the ensemble machine learning framework may comprise a neural network configured to analyze an optimum of one or more time, resource and efficacy variables for the plurality of curated interventions to determine the optimal curated intervention. Method 1300 may proceed by performing one or more steps or operations for presenting (e.g., with the client device) the intervention recommendation for the patient under care to the authorized end user via the graphical user interface of the instance of the end user application (Step 1310).

In accordance with certain aspects of the present disclosure, method 1300 may proceed by performing one or more steps or operations for receiving (e.g., with the client device via the graphical user interface) at least one user-generated input from the authorized end user in response to the intervention recommendation for the patient under care (Step 1312). In certain embodiments, the at least one user-generated input may include outcome data associated with the intervention recommendation. In accordance with certain embodiments, the outcome data may be associated with one or more intervention variables comprising one or more of a qualitative success level, an intervention duration, intervention resources, intervention frequency, intervention intensity and intervention efficacy. In certain embodiments, method 1300 may proceed by performing one or more steps or operations for analyzing (e.g., with the remote server) one or more outcome metrics for the intervention recommendation based on the at least one user-generated input from the authorized end user in response to the intervention recommendation for the patient under care (Step 1314). In certain embodiments, method 1300 may proceed by performing one or more steps or operations for updating or configuring (e.g., with the remote server) a training dataset for the ensemble machine learning framework comprising the outcome data associated with the intervention recommendation (Step 1316).

In accordance with certain embodiments, method 1300 may comprise one or more steps or operations for presenting (e.g., with the client device) a graphical representation of the one or more outcome metrics to the authorized end user via the graphical user interface of the instance of the end user application. In certain embodiments, the ensemble machine learning framework may include one or more of an artificial neural network, a convolutional neural network and a graph neural network. In certain embodiments, the ensemble machine learning framework may comprise a graph-structure data framework comprising a hypergraph, wherein the ensemble machine learning framework comprises the graph neural network. In certain embodiments, the hypergraph may comprise one or more patient behavior and recommended input-output relationships mapped as one or more nodes, vertices and hyperedges on the hypergraph.

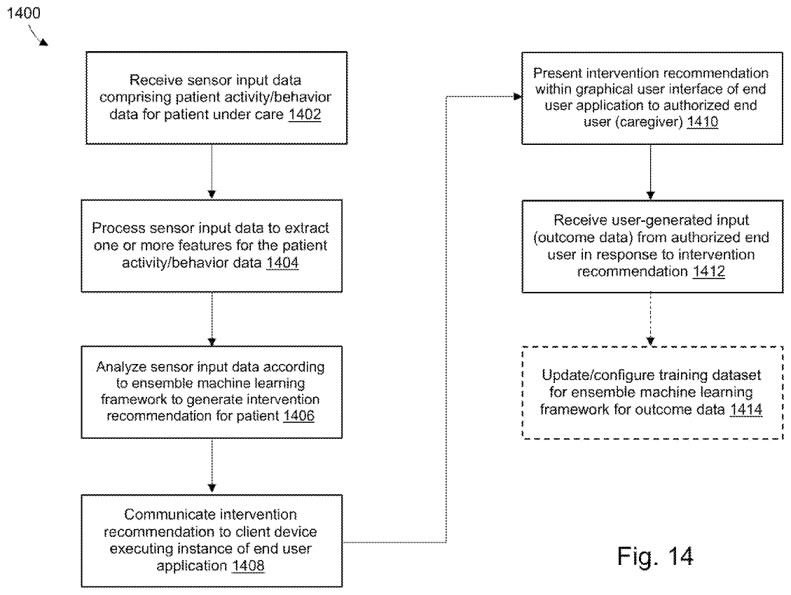

Referring now to FIG. 14, a process flow diagram of a computer-implemented method 1400 for generating a curated medical intervention is shown. Method 1400 may comprise one or more of process steps 1402-1414. In accordance with certain aspects of the present disclosure, method 1400 may be implemented, in whole or in part, within one or more routines of a system for generating a curated medical intervention and/or one or more operations of a computer program product for generating a curated medical intervention, as described herein, and/or may be sequential or successive to one or more steps of method 1300 (as shown and described in FIG. 13).

In accordance with certain aspects of the present disclosure, method 1400 may be initiated by performing one or more steps or operations for receiving (e.g., with a plurality of sensors communicably engaged with a remote server) a plurality of sensor input data comprising a plurality of patient activity data or patient behavior data for a patient under care (Step 1402). In certain embodiments, the plurality of sensors may include one or more camera, physiological sensor, wearable sensor, acoustic sensor and the like. Method 1400 may proceed by performing one or more steps or operations for processing (e.g., with the remote server) the plurality of sensor input data to extract one or more features for the plurality of patient activity data or patient behavior data for the patient under care (Step 1404). In certain embodiments, the one or more features comprise one or more variables in an ensemble machine learning framework. Method 1400 may proceed by performing one or more steps or operations for analyzing (e.g., with the remote server) the plurality of patient activity data or patient behavior data according to the ensemble machine learning framework to generate an intervention recommendation for the patient under care (Step 1406). In certain embodiments, the intervention recommendation may comprise an optimal curated intervention selected from a plurality of curated interventions based on an output of the ensemble machine learning framework. In certain embodiments, the ensemble machine learning framework may comprise an artificial neural network configured to analyze an optimum of one or more time, resource and efficacy variables for the plurality of curated interventions to determine the optimal curated intervention. Method 1400 may proceed by performing one or more steps or operations for communicating (e.g., with the remote server) the intervention recommendation to a client device executing an instance of an end user application (Step 1408). Method 1400 may proceed by performing one or more steps or operations for presenting (e.g., with the client device) the intervention recommendation within a graphical user interface of the end user application to an authorized end user (Step 1410). In certain embodiments, the authorized end user may comprise a caregiver for the patient under care. Method 1400 may include one or more steps or operations for presenting (e.g., with the client device via the graphical user interface) the one or more user-generated inputs comprising a patient selection input and at least one observed patient behavior input (Step 1412). In certain embodiments, the patient selection input may comprise a patient identifier configured to identify the patient under care within an application database communicably engaged with the remote server. In accordance with certain embodiments, method 1400 may include one or more steps or operations for updating or configuring (e.g., with the remote server) a training dataset for the ensemble machine learning framework, the training dataset comprising the outcome data, the one or more user-generated inputs and the sensor input data (Step 1414).

In accordance with certain aspects of the present disclosure, method 1400 may include one or more steps or operations for analyzing (e.g., with the remote server) the one or more user-generated inputs according to the ensemble machine learning framework to generate the intervention recommendation for the patient under care. In accordance with certain embodiments, method 1400 may include one or more steps or operations for analyzing (e.g., with the remote server) the plurality of patient activity data or patient behavior data to generate one or more alert based on one or more configurable threshold comprising one or more of a sensor value, a patient vital and a caregiver input. In accordance with certain embodiments, method 1400 may include one or more steps or operations for receiving (e.g., with the client device via the graphical user interface) at least one user-generated input from the authorized end user in response to the intervention recommendation for the patient under care, the at least one user-generated input comprising outcome data associated with the intervention recommendation. In certain embodiments, the outcome data may comprise one or more intervention variables comprising one or more of a qualitative success level, an intervention duration, intervention resources, intervention frequency, intervention intensity and intervention efficacy.

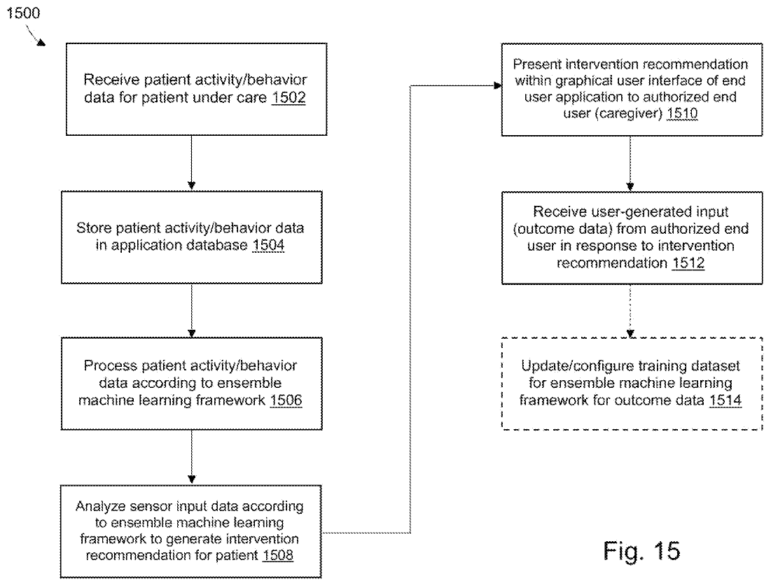

Referring now to FIG. 15, a process flow diagram of a computer-implemented method 1500 for generating a curated medical intervention is shown. Method 1500 may comprise one or more of process steps 1502-1514. In accordance with certain aspects of the present disclosure, method 1500 may be implemented, in whole or in part, within one or more routines of a system for generating a curated medical intervention and/or one or more operations of a computer program product for generating a curated medical intervention, as described herein; and/or may be sequential or successive to one or more steps of method 1300 (as shown and described in FIG. 13); and/or may be sequential or successive to one or more steps of method 1400 (as shown and described in FIG. 14).

In accordance with certain aspects of the present disclosure, method 1500 may be initiated by performing one or more steps or operations for receiving (e.g., with a remote server via an end user device) a plurality of patient activity data or patient behavior data for a patient under care (Step 1502). In certain embodiments, the plurality of patient activity data or patient behavior data may comprise one or more of a plurality of user-generated inputs from an authorized end user via a graphical user interface of an end user application and a plurality of sensor inputs from one or more sensors. In accordance with various aspects of the present disclosure, the authorized end user is a caregiver of the patient under care. Method 1500 may proceed by performing one or more steps or operations for storing (e.g., with an application database communicably engaged with the remote server) the plurality of patient activity data or patient behavior data (Step 1504). Method 1500 may proceed by performing one or more steps or operations for processing (e.g., with the remote server) the plurality of patient activity data or patient behavior data according an ensemble machine learning framework (Step 1506). In certain embodiments, the plurality of patient activity data or patient behavior data may comprise a training dataset for the ensemble machine learning framework, wherein the training dataset is stored in the application database. Method 1500 may proceed by performing one or more steps or operations for analyzing (e.g., with the remote server) the plurality of patient activity data or patient behavior data according the ensemble machine learning framework to generate an intervention recommendation for the patient under care (Step 1508). In certain embodiments, the intervention recommendation may comprise an optimal curated intervention selected from a plurality of curated interventions based on an output of the ensemble machine learning framework. In certain embodiments, the ensemble machine learning framework may comprise a neural network configured to analyze an optimum of one or more time, resource and efficacy variables for the plurality of curated interventions to determine the optimal curated intervention. Method 1500 may proceed by performing one or more steps or operations for presenting (e.g., with the user device) the intervention recommendation for the patient under care to the authorized end user via the graphical user interface of the end user application (Step 1510). In accordance with certain aspects of the present disclosure, method 1500 may further comprise one or more steps or operations for receiving (e.g., with the client device via the graphical user interface) at least one user-generated input from the authorized end user in response to the intervention recommendation for the patient under care (Step 1512). In certain embodiments, the at least one user-generated input may comprise outcome data associated with the intervention recommendation. Method 1500 may further comprise one or more steps or operations for updating or configuring (e.g., with the remote server) a training dataset for the ensemble machine learning framework (Step 1514). In certain embodiments, the training dataset may comprise one or more of the outcome data, the plurality of patient activity data or patient behavior data and the intervention recommendation. In certain embodiments, method 1500 may further comprise one or more steps or operations for analyzing (e.g., with the remote server) one or more outcome metrics for the intervention recommendation based on the outcome data and presenting (e.g., with the client device) a graphical representation of the one or more outcome metrics to the authorized end user via the graphical user interface of the instance of the end user application.

As will be appreciated by one of skill in the art, the present invention may be embodied as a method (including, for example, a computer-implemented process, a business process, and/or any other process), apparatus (including, for example, a system, machine, device, computer program product, and/or the like), or a combination of the foregoing. Accordingly, embodiments of the present invention may take the form of an entirely hardware embodiment, an entirely software embodiment (including firmware, resident software, micro-code, etc.), or an embodiment combining software and hardware aspects that may generally be referred to herein as a “system.” Furthermore, embodiments of the present invention may take the form of a computer program product on a computer-readable medium having computer-executable program code embodied in the medium.

Any suitable transitory or non-transitory computer readable medium may be utilized. The computer readable medium may be, for example but not limited to, an electronic, magnetic, optical, electromagnetic, infrared, or semiconductor system, apparatus, or device. More specific examples of the computer readable medium include, but are not limited to, the following: an electrical connection having one or more wires; a tangible storage medium such as a portable computer diskette, a hard disk, a random-access memory (RAM), a read-only memory (ROM), an erasable programmable read-only memory (EPROM or Flash memory), a compact disc read-only memory (CD-ROM), or other optical or magnetic storage device.

In the context of this document, a computer readable medium may be any medium that can contain, store, communicate, or transport the program for use by or in connection with the instruction execution system, apparatus, or device. The computer usable program code may be transmitted using any appropriate medium, including but not limited to the Internet, wireline, optical fiber cable, radio frequency (RF) signals, or other mediums.

Computer-executable program code for carrying out operations of embodiments of the present invention may be written in an object oriented, scripted, or unscripted programming language such as Java, Perl, Smalltalk, C++, or the like. However, the computer program code for carrying out operations of embodiments of the present invention may also be written in conventional procedural programming languages, such as the “C” programming language or similar programming languages.

Embodiments of the present invention are described above with reference to flowchart illustrations and/or block diagrams of methods, apparatus (systems), and computer program products. It will be understood that each block of the flowchart illustrations and/or block diagrams, and/or combinations of blocks in the flowchart illustrations and/or block diagrams, can be implemented by computer-executable program code portions. These computer-executable program code portions may be provided to a processor of a general-purpose computer, special purpose computer, or other programmable data processing apparatus to produce a particular machine, such that the code portions, which execute via the processor of the computer or other programmable data processing apparatus, create mechanisms for implementing the functions/acts specified in the flowchart and/or block diagram block or blocks.

These computer-executable program code portions may also be stored in a computer-readable memory that can direct a computer or other programmable data processing apparatus to function in a particular manner, such that the code portions stored in the computer readable memory produce an article of manufacture including instruction mechanisms which implement the function/act specified in the flowchart and/or block diagram block(s).

The computer-executable program code may also be loaded onto a computer or other programmable data processing apparatus to cause a series of operational phases to be performed on the computer or other programmable apparatus to produce a computer-implemented process such that the code portions which execute on the computer or other programmable apparatus provide phases for implementing the functions/acts specified in the flowchart and/or block diagram block(s). Alternatively, computer program implemented phases or acts may be combined with operator or human implemented phases or acts in order to carry out an embodiment of the invention.

As the phrase is used herein, a processor may be “configured to” perform a certain function in a variety of ways, including, for example, by having one or more general-purpose circuits perform the function by executing particular computer-executable program code embodied in computer-readable medium, and/or by having one or more application-specific circuits perform the function.

Embodiments of the present invention are described above with reference to flowcharts and/or block diagrams. It will be understood that phases of the processes described herein may be performed in orders different than those illustrated in the flowcharts. In other words, the processes represented by the blocks of a flowchart may, in some embodiments, be in performed in an order other that the order illustrated, may be combined, or divided, or may be performed simultaneously. It will also be understood that the blocks of the block diagrams illustrated, in some embodiments, merely conceptual delineations between systems and one or more of the systems illustrated by a block in the block diagrams may be combined or share hardware and/or software with another one or more of the systems illustrated by a block in the block diagrams. Likewise, a device, system, apparatus, and/or the like may be made up of one or more devices, systems, apparatuses, and/or the like. For example, where a processor is illustrated or described herein, the processor may be made up of a plurality of microprocessors or other processing devices which may or may not be coupled to one another. Likewise, where a memory is illustrated or described herein, the memory may be made up of a plurality of memory devices which may or may not be coupled to one another.

While certain exemplary embodiments have been described and shown in the accompanying drawings, it is to be understood that such embodiments are merely illustrative of, and not restrictive on, the broad invention, and that this invention is not limited to the specific constructions and arrangements shown and described, since various other changes, combinations, omissions, modifications, and substitutions, in addition to those set forth in the above paragraphs, are possible. Those skilled in the art will appreciate that various adaptations and modifications of the just described embodiments can be configured without departing from the scope and spirit of the invention. Therefore, it is to be understood that, within the scope of the appended claims, the invention may be practiced other than as specifically described herein.

What is claimed is:

1. A computer-implemented method for generating a curated medical intervention, comprising:
providing, with a remote server communicably engaged with a client device, an instance of an end user application to the client device, the instance of the end user application comprising a graphical user interface rendered at a display of the client device, wherein the instance of the end user application is instantiated by an authorized end user of the end user application, the authorized end user comprising a caregiver for a patient under care;
receiving, with the client device, one or more user-generated inputs from the authorized end user via the graphical user interface, the one or more user-generated inputs comprising a patient selection input and at least one observed patient behavior input, wherein the patient selection input comprises a patient identifier configured to identify the patient under care within an application database communicably engaged with the remote server;
processing, with the remote server or the client device, the one or more user-generated inputs to determine one or more variables associated with the patient selection input and the at least one observed patient behavior input;
analyzing, with the remote server, the one or more user-generated inputs according to an ensemble machine learning framework to generate an intervention recommendation for the patient under care, the intervention recommendation comprising an optimal curated intervention selected from a plurality of curated interventions based on an output of the ensemble machine learning framework, wherein the ensemble machine learning framework comprises a neural network configured to analyze an optimum of one or more time, resource and efficacy variables for the plurality of curated interventions to determine the optimal curated intervention; and
presenting, with the client device, the intervention recommendation for the patient under care to the authorized end user via the graphical user interface of the instance of the end user application.

2. The method of claim 1 further comprising receiving, with the client device via the graphical user interface, at least one user-generated input from the authorized end user in response to the intervention recommendation for the patient under care, the at least one user-generated input comprising outcome data associated with the intervention recommendation.

3. The method of claim 2 wherein the outcome data is associated with one or more intervention variables comprising one or more of a qualitative success level, an intervention duration, intervention resources, intervention frequency, intervention intensity and intervention efficacy.

4. The method of claim 3 further comprising updating or configuring, with the remote server, a training dataset for the ensemble machine learning framework comprising the outcome data associated with the intervention recommendation.

5. The method of claim 2 further comprising analyzing, with the remote server, one or more outcome metrics for the intervention recommendation based on the at least one user-generated input from the authorized end user in response to the intervention recommendation for the patient under care.

6. The method of claim 5 further comprising presenting, with the client device, a graphical representation of the one or more outcome metrics to the authorized end user via the graphical user interface of the instance of the end user application.

7. The method of claim 1 wherein the ensemble machine learning framework comprises one or more of an artificial neural network, a convolutional neural network and a graph neural network.

8. The method of claim 7 wherein the ensemble machine learning framework comprises a graph-structure data framework comprising a hypergraph, wherein the ensemble machine learning framework comprises the graph neural network.

9. The method of claim 8 wherein the hypergraph comprises one or more patient behavior and recommended input-output relationships mapped as one or more nodes, vertices and hyperedges on the hypergraph.

10. A computer-implemented method for generating a curated medical intervention, comprising:
receiving, with a plurality of sensors communicably engaged with a remote server, a plurality of sensor input data comprising a plurality of patient activity data or patient behavior data for a patient under care, the plurality of sensors comprising one or more of a camera, a physiological sensor, a wearable sensor and an acoustic sensor;
processing, with the remote server, the plurality of sensor input data to extract one or more features for the plurality of patient activity data or patient behavior data for the patient under care, wherein the one or more features comprise one or more variables in an ensemble machine learning framework;
analyzing, with the remote server, the plurality of patient activity data or patient behavior data according to the ensemble machine learning framework to generate an intervention recommendation for the patient under care, the intervention recommendation comprising an optimal curated intervention selected from a plurality of curated interventions based on an output of the ensemble machine learning framework, wherein the ensemble machine learning framework comprises a neural network configured to analyze an optimum of one or more time, resource and efficacy variables for the plurality of curated interventions to determine the optimal curated intervention;
communicating, with the remote server, the intervention recommendation to a client device executing an instance of an end user application; and
presenting, with the client device, the intervention recommendation within a graphical user interface of the end user application to an authorized end user, wherein the authorized end user comprises a caregiver for the patient under care.

11. The method of claim 10 further comprising receiving, with the client device via the graphical user interface, the one or more user-generated inputs comprising a patient selection input and at least one observed patient behavior input, wherein the patient selection input comprises a patient identifier configured to identify the patient under care within an application database communicably engaged with the remote server.

12. The method of claim 11 further comprising analyzing, with the remote server, the one or more user-generated inputs according to the ensemble machine learning framework to generate the intervention recommendation for the patient under care.

13. The method of claim 10 further comprising analyzing, with the remote server, the plurality of patient activity data or patient behavior data to generate one or more alert based on one or more configurable threshold comprising one or more of a sensor value, a patient vital and a caregiver input.

14. The method of claim 12 further comprising receiving, with the client device via the graphical user interface, at least one user-generated input from the authorized end user in response to the intervention recommendation for the patient under care, the at least one user-generated input comprising outcome data associated with the intervention recommendation.

15. The method of claim 14 wherein the outcome data comprises one or more intervention variables comprising one or more of a qualitative success level, an intervention duration, intervention resources, intervention frequency, intervention intensity and intervention efficacy.

16. The method of claim 15 further comprising updating or configuring, with the remote server, a training dataset for the ensemble machine learning framework, the training dataset comprising the outcome data, the one or more user-generated inputs and the sensor input data.

17. A computer-implemented method for generating a curated medical intervention, comprising:
receiving, with a remote server via an end user device, a plurality of patient activity data or patient behavior data for a patient under care, the plurality of patient activity data or patient behavior data comprising one or more of a plurality of user-generated inputs from an authorized end user via a graphical user interface of an end user application and a plurality of sensor inputs from one or more sensors, wherein the authorized end user comprises a caregiver of the patient under care;
storing, with an application database communicably engaged with the remote server, the plurality of patient activity data or patient behavior data;
processing, with the remote server, the plurality of patient activity data or patient behavior data according to an ensemble machine learning framework, wherein the plurality of patient activity data or patient behavior data comprises a training dataset for the ensemble machine learning framework, wherein the training dataset is stored in the application database;
analyzing, with the remote server, the plurality of patient activity data or patient behavior data according the ensemble machine learning framework to generate an intervention recommendation for the patient under care, the intervention recommendation comprising an optimal curated intervention selected from a plurality of curated interventions based on an output of the ensemble machine learning framework, wherein the ensemble machine learning framework comprises a neural network configured to analyze an optimum of one or more time, resource and efficacy variables for the plurality of curated interventions to determine the optimal curated intervention; and
presenting, with the user device, the intervention recommendation for the patient under care to the authorized end user via the graphical user interface of the end user application.

18. The method of claim 17 further comprising receiving, with the client device via the graphical user interface, at least one user-generated input from the authorized end user in response to the intervention recommendation for the patient under care, the at least one user-generated input comprising outcome data associated with the intervention recommendation.

19. The method of claim 18 further comprising updating or configuring, with the remote server, the training dataset for the ensemble machine learning framework, the training dataset comprising the outcome data, the plurality of patient activity data or patient behavior data and the intervention recommendation.

20. The method of claim 18 further comprising:
analyzing, with the remote server, one or more outcome metrics for the intervention recommendation based on the outcome data; and
presenting, with the client device, a graphical representation of the one or more outcome metrics to the authorized end user via the graphical user interface of the instance of the end user application.
